(19) United States
(12) Patent Application Publication
(10) Pub. No.: US 2026/0345886 A1
(43) Pub. Date: Nov. 5, 2026

(54) METHOD AND SYSTEM FOR REMOVING BICARBONATE FROM DIALYSATE

(71) Applicant: HemoCleanse Technologies, LLC, Lafayette, IN (US)

(72) Inventors: Stephen Ash, Lafayette, IN (US); David Carr, Lafayette, IN (US)

(73) Assignee: HemoCleanse Technologies, LLC, Lafayette, IN (US)

(21) Appl. No.: 19/327,512

(22) Filed: Sep. 12, 2025

Related U.S. Application Data
(60) Provisional application No. 63/693,971, filed on Sep. 12, 2024.

(51) Int. Cl. A61M 1/34; A61M 1/36

(52) U.S. Cl. CPC A61M 1/3448; A61M 1/3633; A61M 2205/3324

(57) ABSTRACT

A sorbent system for bicarbonate removal from dialysate from a patient. More particularly, embodiments of the invention provide a sorbent system that removes bicarbonate from dialysate using calcium hydroxide. The system can pump a dialysate through the bicarbonate removal system using a first pump. A a second pump can pump a sample amount of dialysate to CO2 measurement system that can be used to determine how much calcium hydroxide must be added to the dialysate to a reactor with a mixer to mix the dialysate and the calcium hydroxide. The dialysate can pass through a filter to remove resulting calcium carbonate before being returned to the dialyzer. One or more additional systems can ensure proper pH and volume of the dialysate.

### CROSS-REFERENCE TO RELATED PATENTS APPLICATIONS

This U.S. Patent Application is U.S. Nonprovisional Application Number, which claims priority to U.S. Provisional Application No. 63/693,971 filed Sep. 12, 2024, to the above-named inventors, the disclosure of which is considered part of the disclosure of this application and is hereby incorporated by reference in its entirety.

### FIELD OF THE INVENTION

This invention relates generally to a sorbent system that removes bicarbonate from dialysate. More particularly, embodiments of the invention provide a sorbent system that removes bicarbonate from dialysate using calcium hydroxide.

### BACKGROUND

It is often medically necessary to place a patient on a respirator after the patient has experienced severe respiratory failure. Most patients are placed on respirators because of hypercapnia and respiratory acidosis. Many of these patients have adequate oxygenation, through high FiO₂ and frequent inspiration. Patients with severe pneumonia or Acute respiratory distress syndrome (ARDS) can quickly worsen when placed on a respirator; mortality rate is over 60% of patients for Covid-19 pneumonia when on a respirator. Extracorporeal Membrane Oxygenation (ECMO) is the treatment of last resort in COVID-19 patients, but up to 80% of these patients die. In all patients, ECMO and ECCO₂R are difficult, intensive treatments with complications of vascular damage from large catheters, bleeding, infection, and cerebral ischemia.

A potential solution is to remove CO₂ by dialysis. In respiratory failure, plasma bicarbonate increases to counter the acidosis, as does bicarbonate inside red cells (which can transfer to plasma quickly). With a plasma bicarbonate of 30 meq/L and 400 ml/min blood flow rate, a dialyzer could transfer 12 meq/min of bicarbonate to dialysate. Each meq of bicarbonate removed equals one mM of CO₂ removed (25 ml of CO₂ gas). Normal human total CO₂ generation is 10 mM/min (250 ml/min of CO₂ gas). Therefore, a standard hemodialysis or CVVHD system could remove enough CO2 in a patient with hypercapnic respiratory failure to avoid the need for respiratory therapy.

However, dialysis and CVVHD as performed today also have their problems. Clotting of fibers in the dialyzers means that blood side systems must be replaced frequently. In kidney failure patients on CVVHD the actual treatment duration for each pack is less than 24 hours. In COVID-19 patients with kidney failure coagulopathy results in clotting and filter duration is only 8-12 hours. Removal of sufficient bicarbonate by dialysis requires that the bicarbonate concentration in the dialysate is near to zero (0). To avoid sudden severe acidosis and maintain anion levels, another base has to be added to the dialysate to replace the bicarbonate. Using NaOH as a base in dialysate creates very high pH in the blood and returns huge amounts of Na+to the patient. Other bases such as lactate and citrate rely on liver metabolism to create bicarbonate, and this is not reliably predictable.

There is therefore a need for new and improved methods and systems for removing bicarbonate from dialysate and replaces it with CL⁻ while avoiding obstructions by clots. The present disclosure can fulfill an unmet need by providing Extracorporeal carbon dioxide removal (ECCO₂R) with a dialyzer that avoids obstruction by clots, and a sorbent system that removes bicarbonate from dialysate and replaces it with Cl⁻ and OH⁻ without returning any cations (such as Na⁺ or Ca⁺⁺).

### BRIEF SUMMARY OF THE INVENTION

The present invention is a method and system for bicarbonate removal from dialysate. In one aspect the present invention is a sorbent system to remove HCO₃⁻ from dialysate for extracorporeal CO₂ Removal (ECCO₂R). The system and method for sufficient bicarbonate removal to function as Extracorporeal carbon dioxide removal (ECCO₂R) system. Bicarbonate removal equals CO₂ generation by a normal human and far exceeds that of prior systems attempting bicarbonate removal with low bicarbonate dialysate. In vitro, the reaction of Ca(OH)₂ with HCO₃⁻ to form CaCO₃ is instantaneous, and there is no need to provide time within a continuous stirred tank reactor (CSTR) to complete the reaction. Particle collector options can include continuous flow centrifuge with disposable bowls. Alternatively, the dialysate can be passed through a microporous membrane filter is a simpler and probably less expensive method for collecting and disposing of the collected particle. In vitro studies have shown that it is necessary to always have a little bicarbonate in the dialysate after Ca(OH)₂ reaction, to prevent release of Ca⁺⁺. This requires monitoring of total CO₂ levels in inflow and outflow dialysate (or monitoring outflow Ca⁺⁺ level).

The present disclosure further relates to a method of removal of bicarbonate from a dialysate by first pumping a dialysate through the bicarbonate removal system of the present disclosure having a dialyzer and using a first pump, wherein a second pump provides a first specimen of the dialysate to the CO₂ measurement system, wherein the CO₂ measurement system provides a CO₂ measurement of the dialysate to the calcium hydroxide system. The remaining dialysate can be pumped into a mixer. A prescribed amount of calcium hydroxide from the calcium hydroxide system can be provided or pumped into the mixer based upon the CO₂ measurements at the CO₂ measurement system. The dialysate and calcium hydroxide can be continuously mixed withing a reactor until a calcium carbonate precipitate forms. The dialysate can be pumped through a filter to remove any unreacted calcium hydroxide and calcium carbonate precipitate. The pH of the dialysate can then be measured to determine a pH reading with the pH detector. The system can optionally utilize a volume control system to remove any excess volume of dialysate. A safety monitoring system can be used to further analyze the dialysate. The system can have a pH electrode to measure the pH prior to being pumped back into the dialyzer. An acid can be added to the dialysate to reduce pH of the dialysate if the pH reading is 12.5 or higher. The dialysate can be pumped back into the into the dialyzer and out to the patient.

In yet another aspect, the disclosure provides a system for removing bicarbonate from dialysate. The system can include a dialyzer having an inlet and an outlet, further comprising one or more conduits providing a loop between the inlet and outlet for the dialysate to flow. A dialysate pump positioned after the dialyzer can pump the dialysate through the loop of the system. A first sample pump can remove a pre-determined sample amount of dialysate from the dialyzer loop. The sample pump can be communicatively coupled to a CO₂ measurement system, wherein the CO₂ measurement system can be used to determine the total CO₂ in the dialysate. A calcium hydroxide dispensing system can be communicatively coupled to the CO₂ measurement system. The calcium hydroxide dispensing system includes a calcium hydroxide pump and a hopper portion, wherein the hopper portion can store a calcium hydroxide powder or suspension at a pre-determined concentration, wherein the calcium hydroxide dispensing system is communicatively coupled to the CO₂ measurement system. The system can include a mixer and/or reactor system that can be fluidly coupled to the calcium hydroxide dispensing system. The dialysate can be pumped to a reactor with a mixer to vigorously mix the dialysate and the calcium hydroxide. A volume control system comprising a volume control pump and a volume waste reservoir can remove a prescribed amount of volume from the loop of the system. The system can use a safety monitor system comprising one or more sensors to monitor the ion components of the dialysate. A pH sensor of the system to obtain an outlet pH of the dialysate. The system can include an acid pump system communicatively coupled to the pH sensor to provide an acid to the dialysate when the dialysate is at or above a prescribed pH.

The invention now will be described more fully hereinafter with reference to the accompanying drawings, which are intended to be read in conjunction with both this summary, the detailed description and any preferred and/or particular embodiments specifically discussed or otherwise disclosed. This invention may, however, be embodied in many different forms and should not be construed as limited to the embodiments set forth herein; rather, these embodiments are provided by way of illustration only and so that this disclosure will be thorough, complete and will fully convey the full scope of the invention to those skilled in the art.

### BRIEF DESCRIPTION OF THE DRAWINGS

FIG. 1 is a schematic illustration of the Extracorporeal carbon dioxide removal (ECCO₂R) with a dialyzer that avoids obstruction by clots, and a sorbent system that removes bicarbonate from dialysate and replaces it with a mixture of Cl⁻ and OH⁻, no cations (such as Na⁺ or Ca⁺⁺) are returned to blood, according to the present disclosure.

FIG. 2 is a schematic illustration of the experimental setup for bicarbonate removal from dialysate, according to the present disclosure.

FIG. 3 is schematic of an exemplary embodiment of a bicarbonate removal system of the present disclosure.

FIG. 4 is a graphical illustration of the Ca(OH)₂ capacity for binding bicarbonate from solution (expressed as mM CO₂).

FIG. 5 is a graphical illustration of the total CO₂ during Bicarb removal with Ca(OH)₂ in static mixer, according to the present disclosure.

FIG. 6 is a graphical illustration of total Calcium and soluble Calcium during Bicarb removal with Ca(OH)₂ in static mixer, according to the present disclosure.

FIG. 7 is a table illustration of the specifications of sorbent system for bicarbonate removal from dialysate using Ca(OH)₂, removing 100% of generated CO₂ in 12 hours, according to the present disclosure.

### DETAILED DESCRIPTION OF THE INVENTION

The following detailed description includes references to the accompanying drawings, which forms a part of the detailed description. The drawings show, by way of illustration, specific embodiments in which the invention may be practiced. These embodiments, which are also referred to herein as “examples,” are described in enough detail to enable those skilled in the art to practice the invention. The embodiments may be combined, other embodiments may be utilized, or structural, and logical changes may be made without departing from the scope of the present invention. The following detailed description is, therefore, not to be taken in a limiting sense.

Before the present invention of this disclosure is described in such detail, however, it is to be understood that this invention is not limited to particular variations set forth and may, of course, vary. Various changes may be made to the invention described and equivalents may be substituted without departing from the true spirit and scope of the invention. In addition, many modifications may be made to adapt a particular situation, material, composition of matter, process, process act(s), or step(s), to the objective(s), spirit, or scope of the present invention. All such modifications are intended to be within the scope of the disclosure made herein.

Unless otherwise indicated, the words and phrases presented in this document have their ordinary meanings to one of skill in the art. Such ordinary meanings can be obtained by reference to their use in the art and by reference to general and scientific dictionaries.

References in the specification to “one embodiment” indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The following explanations of certain terms are meant to be illustrative rather than exhaustive. These terms have their ordinary meanings given by usage in the art and in addition include the following explanations.

As used herein, the term “and/or” refers to any one of the items, any combination of the items, or all of the items with which this term is associated.

As used herein, the singular forms “a,” “an,” and “the” include plural reference unless the context clearly dictates otherwise.

As used herein, the terms “include,” “for example,” “such as,” and the like are used illustratively and are not intended to limit the present invention.

As used herein, the terms “preferred” and “preferably” refer to embodiments of the invention that may afford certain benefits, under certain circumstances. However, other embodiments may also be preferred, under the same or other circumstances.

Furthermore, the recitation of one or more preferred embodiments does not imply that other embodiments are not useful and is not intended to exclude other embodiments from the scope of the invention.

As used herein, the terms “front,” “back,” “rear,” “upper,” “lower,” “right,” and “left” in this description are merely used to identify the various elements as they are oriented in the FIGS, with “front,” “back,” and “rear” being relative to the apparatus. These terms are not meant to limit the elements that they describe, as the various elements may be oriented differently in various applications.

As used herein, the term “coupled” means the joining of two members directly or indirectly to one another. Such joining may be stationary in nature or movable in nature. Such joining may be achieved with the two members or the two members and any additional intermediate members being integrally formed as a single unitary body with one another or with the two members or the two members and any additional intermediate members being attached to one another. Such joining may be permanent in nature or alternatively may be removable or releasable in nature. Similarly, coupled can refer to a two member or elements being in communicatively coupled, wherein the two elements may be electronically, through various means, such as a metallic wire, wireless network, optical fiber, or other medium and methods.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element without departing from the teachings of the disclosure.

Within the specification the following specific definitions or terms may be used: ARDS: Acute Respiratory Distress Syndrome; CSTR: Continuous Stirred Tank Reactor. ECCO₂R: Extracorporeal carbon dioxide removal; ECMO: Extracorporeal Membrane Oxygenation; Means: A means of doing or implementing an action, feature, and so on, known, or obvious to those skilled in the art.

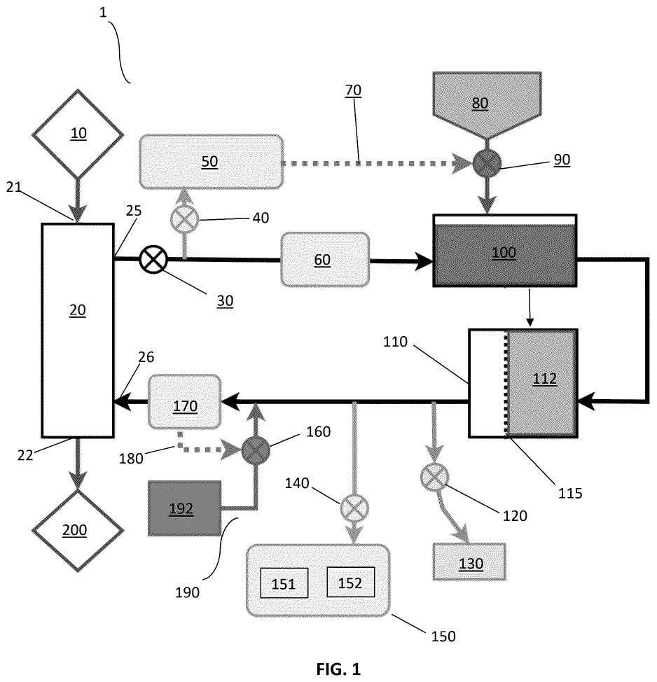

Referring now to FIG. 1-7 , the present disclosure relates to a system and method for bicarbonate removal including but not limited to a system for extracorporeal blood treatment system to generate dialysate. The present disclosure utilizes an infusion of a suspension of calcium hydroxide into the flowing dialysate, which can operate by combining with the bicarbonate to form calcium carbonate. The particles of calcium carbonate can then be collected in a filter, to remove them from the flowing dialysate. In some exemplary embodiments, a continuous centrifuge system could be utilized to capture the particles. The system further includes an infusion system to correct the very high pH of the dialysate caused by the calcium hydroxide by infusing HCl. The HCl can replace the hydroxide anions with chloride.

As shown in FIG. 1, one exemplary embodiment a system 1 of the present disclosure can introduce blood in 10 from a patient into a dialyzer 20 which can have an inlet 21 and an outlet 22 that provides blood 10 from a patient to the dialyzer and blood 200 from the dialyzer back to the patient. Similarly, the dialyzer can have dialysate outlet 25 and a dialysate inlet 26. A dialysate pump 30 can be used to pump the initial dialysate from the dialyzer through the system loop. The various components of the system can be fluidly connected through one o or more conduits or tubes. The system can additionally include a carbon dioxide (CO₂) measurement system 50 that can measure the bicarbonate amount in the dialysate using one or more sensors as it enters the system 50.

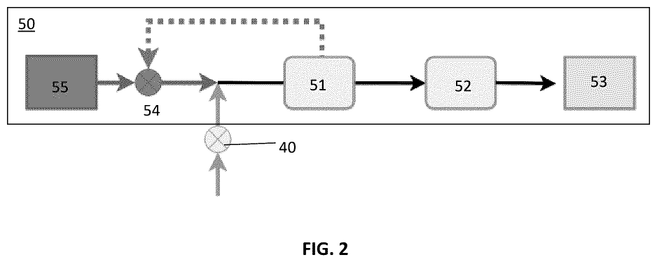

As shown in FIG. 2, some exemplary embodiments of a CO₂ measurement system 50 can include a pH electrode 51, CO₂ electrode 52, a waste reservoir 53, an acid reservoir 55 and acid pump 54. A sample pump 40 can pump a pre-determined amount or for a prescribed duration of the dialysate from the loop to the CO₂ measurement system 50. The CO₂ measurement system 50 can be communicatively coupled to a Ca(OH)₂ dispenser system 80. In some exemplary embodiments, the dialysate should be acidified to a pH between about 4.8 to about 5.2 to convert all bicarbonate to CO₂. A feedback control system using an acid pump can be used to properly acidify the dialysate sample for the CO₂ electrode of the CO₂ measurement system 50. The first specimen or sample pumped to the CO₂ measurement system 50 can be further discarded after the analysis is conducted or it be returned to the dialysate circuit, as shown in FIG. 1.

The dialysate can further pass through a blood leak detection system 60 of the system 1 that can be used to monitor the dialysate outflow. The blood leak detection system 60 can utilize any suitable method including but not limited to an optical sensor. The blood leak detection system 60 can be positioned prior to the mixer reactor 100. The dialysate can flow through the blood leak detection system 60 and into the mixer reactor 100. The mixer reactor can be fluidly coupled to the Ca(OH)₂ dispenser system 80.

A Ca(OH)₂ dispenser system 80 can include a pump 90, dispenser, or other means to provide a prescribed amount of Ca(OH)₂ into a mixer or reactor 100. In some exemplary embodiments, the Ca(OH)₂ dispenser system can include a tank or storage container of Ca(OH)₂, such as a Ca(OH)₂ suspension or Ca(OH)₂ powder. In one exemplary embodiment, the Ca(OH)₂ provided can be a concentrated suspension of Ca(OH)₂ which can then be vigorously mixed to make a uniform suspension with the dialysate. The Ca(OH)₂ dispenser system 80 can use a flow meter, scale, or other measurement device to monitor the amount of Ca(OH)₂ that is dispensed into the dialysate at the mixer 100. In some exemplary embodiments, the mixer reactor 100 can be a static mixer used to combine the dialysate with sodium hydroxide. The amount of Ca(OH)₂ provided by the Ca(OH)₂ dispenser system 80 can be controlled based primarily on the incoming total CO₂ measured at the CO₂ monitoring system 50, or the outflow total CO₂ and Calcium measured at the safety monitor system 150 to determine the amount of Ca(OH)₂ provided by the pump 90. The safety monitor system 150 can have a similar configuration as the inlet CO₂ measurement system 50 shown in FIG. 2. The various monitoring and dispensing systems can be communicatively coupled to allow for the system to optimize the properties of the dialysate.

Based upon the readings from the CO₂ system 50 the Ca(OH)₂ dispenser system 80 can initiate the dispensing of Ca(OH)₂ into the mixer reactor 100. The bicarbonate withing the dialysate can then be mixed the prescribed amount of calcium hydroxide as provided to the mixer reactor based upon the total carbon dioxide (“total CO₂”) measurement by the CO₂ measurement system 50. As the dialysate and calcium hydroxide react within the mixer reactor 100, the bicarbonate will be precipitated from the dialysate as calcium carbonate.

In some exemplary embodiments, the mixer reactor 100 can include a mixer portion and a reactor portion. In other exemplary embodiments, the mixer reactor can be a static mixer 100 and the dialysate can be passed into a reactor 110. The reactor 110 can take any suitable form, but in some exemplary embodiments, the reactor 110 can be a continuous stirred tank reactor 110. The following reaction occurs at the reactor 110: Ca(OH)2+HCO3−=CaCO3+H2O+OH− to produce a calcium carbonate precipitate. In some exemplary embodiments one mole of hydroxide can be produced for every mole of bicarb removed. Calcium carbonate is an insoluble precipitate produced that can then be separated from the dialysate using any filter means including filtration, centrifugation, or both. In some exemplary embodiments, the reactor 110 further includes a filter 115 incorporated or as a separate system to filter the calcium carbonate 112 from the dialysate to ensure that it does not return back to the patient. The reactor size and mixing are designed to promote complete reaction of the dialysate CO₂.

In some exemplary embodiments, the filter 115 downstream of the reactor can prevent calcium carbonate and calcium hydroxide particles from returning to the dialyzer 20 where they would eventually accumulate on the dialysate side of the membranes and destroy the dialyzer function. In some exemplary embodiments, the filter can be a membrane filter with a pore size between about 0.2-2.5 microns. In one exemplary embodiment, a filter can have about 0.22 μm pores which have been shown to remove virtually all particulates that can be detected by optical absorbance. Because the dialysate will need to be sterile, a bacterial retentive filter of 0.22 μm or smaller pore size may be desirable. The most common commercially available membranes seem to be 0.22 μm. The solids filter must have capacity for the CaCO3 produced by the bicarbonate removal, namely 700 grams per 12 hours. The Millipore Opticap 10 XL with 0.22 μm Durapore, 0.69 m2 . has capacity for over 500 grams CaCO3. This filter capsule has a pleated membrane. The next larger size filter capsule, Opticap 20 XL has surface area =1.4 m2 which should be adequate for containing the calcium carbonate with modest pressure drop. Alternative filter designs with more space between membranes for accumulation of particulates would need much less membrane area (approximately 0.2 m2). In some exemplary embodiments, a filter of the present disclosure can have a capacity for approximately 9 hours of use is shown. In some exemplary embodiments, the CaCO₃ filter can include a large thin-layer filter with effective pore size less than about 10 microns and can remove calcium solids and keep soluble [Ca] at very low level. It should be understood that other suitable filter sizes may be used depending upon the application and solids content.

In some exemplary embodiments, the reactor can further include a waste system that can include a volume control pump and a waste reservoir or waste conduit to remove excess dialysate that may be generated. The waste pump 120 can remove a prescribed amount of dialysate from the loop and into a waste reservoir 130 or external reservoir. The waste pump can be used to ensure that the patient's blood volume can be maintained close to the volume before treatment is started with the system 1. The volume control pump 120 can remove the volume of added calcium hydroxide suspension plus added HCl, minus the volume sampled for [CO2] and [Ca] measurement. Therapeutic net removal or addition of blood volume is also possible.

Optionally, in some exemplary embodiments, the system 1 can further include a second sample pump 140 that can remove a prescribed specimen or sample from the dialysate after the reactor. The second specimen can be provided to a safety monitor system 150 that can include one or more sensors which can include but are not limited to a CO₂ sensor 151 and a calcium sensor 152. The safety monitor system 150 can obtain a measurement of CO₂ and calcium present in the dialysate. The safety monitor system 150 can measure total CO₂ of the reactor output, operating the same as the inlet CO₂ electrode. CO₂ in the reactor output is not desirable and indicates that the Ca(OH)₂ dose should be increased. The fluid used for analysis of total CO₂ is acidified and is a small amount, so it is directed to the waste rather than returned to the main dialysate stream. Similarly, the calcium sensor 152 can measure calcium in the dialysate returning to patient. This can provide a safety back-up monitor of the system 1. In order to not deplete the patient of calcium and also not administer high doses of calcium, the returning dialysate should ideally have [Ca] between about 50% and 100% of normal plasma ionized [Ca]. Excess [Ca] indicates that the Ca(OH)2 dose should be decreased by the system 1.

The system 1 of the present disclosure can further include an acidifier system 190 that can include a pump 160 that can pump acid from the acidifier system 190 to the dialysate. The acidifier system can include a reservoir 192 for holding an acid and the pump 160. The acidifier system 190 can further be communicatively coupled 180 to a pH electrode or measuring system 170. The pH electrode can signal to the acidifier system 190 and/or pump directly to provide an acid to the dialysate. In some exemplary embodiments wherein the desired final dialysate pH is less than or equal to about 11.0, the amount of acid necessary to achieve this will be close to the amount of calcium hydroxide used in the process, namely at least about 200 mEq acid/hour when Ca(OH)2 dose is about 250 mEq/hr. In some exemplary embodiment, HCl can be the preferred acid as it can neutralize the excess hydroxide and replaces it with chloride. In order to keep the HCl volume reasonable, an HCl solution can be between about 4 M to 6 M. The acidifier system 190 and pH electrode may be required to prevent alkaline hemolysis of the patient's blood as the binding of bicarbonate to calcium hydroxide raises the dialysate pH to 12.5 or greater, which is not compatible with blood. In some exemplary embodiments, the target pH for dialysate that is processed through the system loop should be a pH 10 or less.

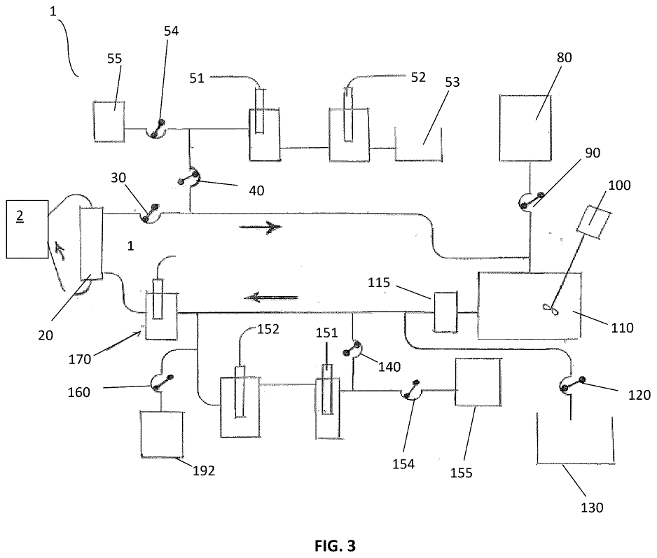

FIG. 3 provides an illustration of another exemplary embodiment of the system/apparatus 1 of the present disclosure for removal of bicarbonate from a patient 2. The system 1 can include a dialyzer 20 and a pump 30 to pump the dialysate from the dialyzer through system loop shown as diagramed with the directional arrows. A first sample pump 40 can provide dialysate to a first inlet measurement system 50 that can include a pH electrode 51 and a CO2 electrode 52 and a waste reservoir 53. An acid reservoir 55 having an acid that can be pumped by an acid pump 54 to acidify the dialysate sample obtained by the first sample pump 40. The main dialysate can further pass to the reactor 110 where calcium hydroxide can be added to the reactor 110 based upon the measurements obtained at the first inlet measurement system 50. A mixer 100 can be used to vigorously mix the dialysate and the calcium carbonate. The mixed dialysate can then pass through a filter 115. A waste pump 120 can remove excess dialysate to a waste reservoir 130. A second measurement pump 140 can obtain a second sample from the dialysate to obtain various measurements using a calcium probe or electrode, pH electrode 151, and/or CO2 electrode 152. A similar acid pump 154 and reservoir 155 can be used to acidify the sample. A final pH electrode 170 can be used to obtain a final blood compatible pH measurement. A separate acid pump 160 and reservoir 192 can be used to provide an acid to the dialysate before it pumped back into the dialyzer 20.

In some exemplary embodiments, a system of the present disclosure can include a hemodialysis system to generate a dialysate from which bicarbonate can be removed. A stirred tank reactor system can be provided where bicarb can be removed from the dialysate. One or more CO₂ electrodes with pH adjustment systems can be provided to measure bicarbonate in and out of the reactor. A calcium hydroxide delivery subsystem can be provided. A system to adjust pH of dialysate returning to the patient can further be included in the system of the present disclosure. The system can further include a waste pump to maintain proper patient hydration during the operation of the system.

The method of the present disclosure avoids release of way too much calcium to the dialysate. By adjusting the mixture rate of calcium hydroxide to dialysate, so that almost all the calcium hydroxide is converted to calcium carbonate, the calcium level decreases dramatically from where it was in the suspension of calcium hydroxide. If the amount of the bicarbonate is known in the dialysate and the added calcium hydroxide equals the moles of the measured bicarbonate plus or minus about 5%, the resultant dialysate mixture will have the highest bicarbonate removal and minimum calcium at equilibrium. The present disclosure also provides a method of removing bicarbonate from a patient's blood utilizing the system 1 described herein.

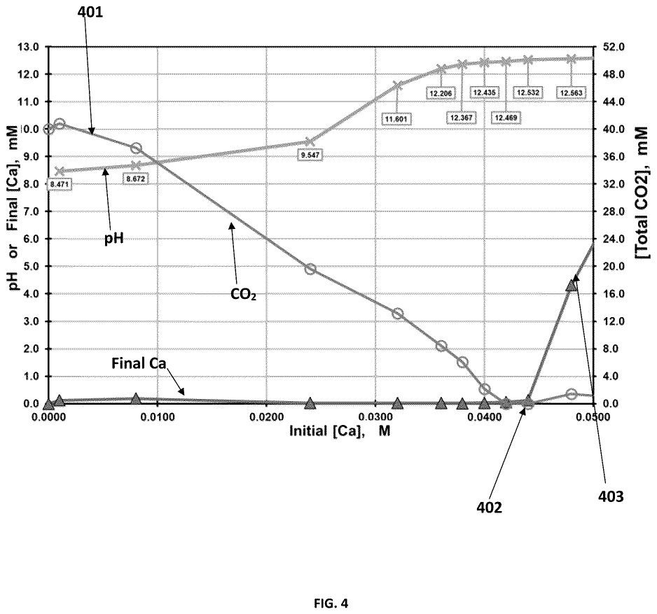

FIG. 4 provides a graphical illustration of the Ca(OH)₂ capacity: As Ca(OH)₂ is added to a solution of HCO₃⁻ it forms CaCO₃ until all the HCO₃⁻ is removed (See 401). When the moles of Ca(OH)₂ added exceed the moles of HCO₃⁻ in the solution, then ionized Ca⁺⁺ rises quickly (See 403). The 1:1 molar binding means 1 gram of Ca(OH)₂ binds 13 mEq of HCO₃⁻ (=1.25 L of CO₂). (See 402)

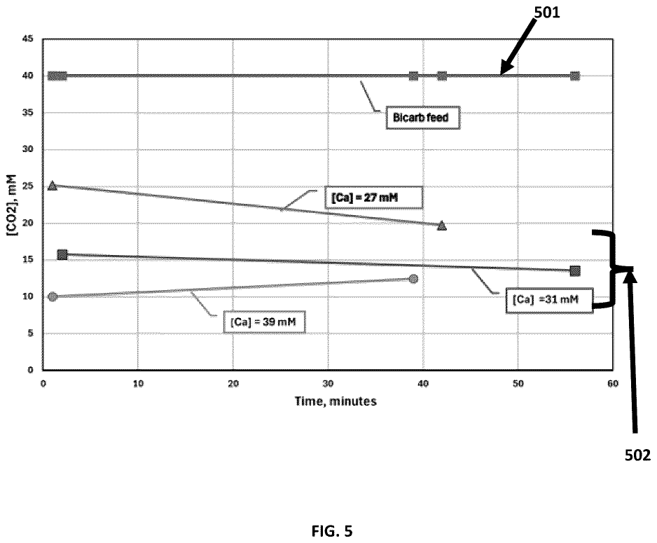

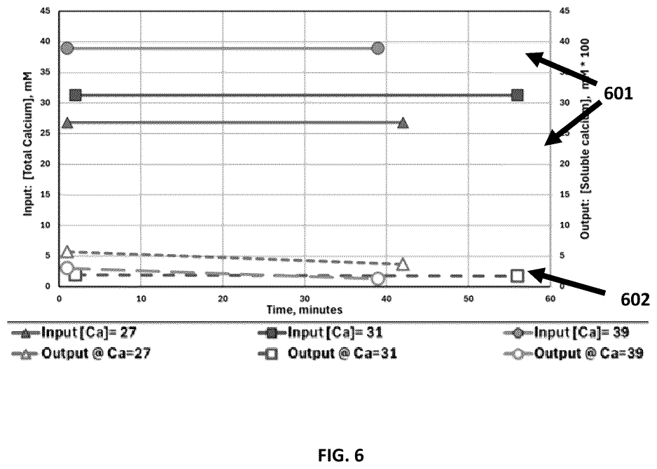

FIG. 5 provides the total CO2 during bicarbonate removal with the calcium hydroxide in a static mixer. The inflow of HCO3 can be about 40 mM (see 501). As the increasing molar rate of infusion of the calcium hydroxide the effluent HCO3 goes lower and lower(See 502). The concentration of Ca++ in the dialysate stays below physiological levels as shown in FIG. 6. The inflow of calcium hydroxide levels can be about 25-39 mM as shown at 601. The outflow of ionized Ca++ levels all are less than about 0.05 nM as shown at 602.

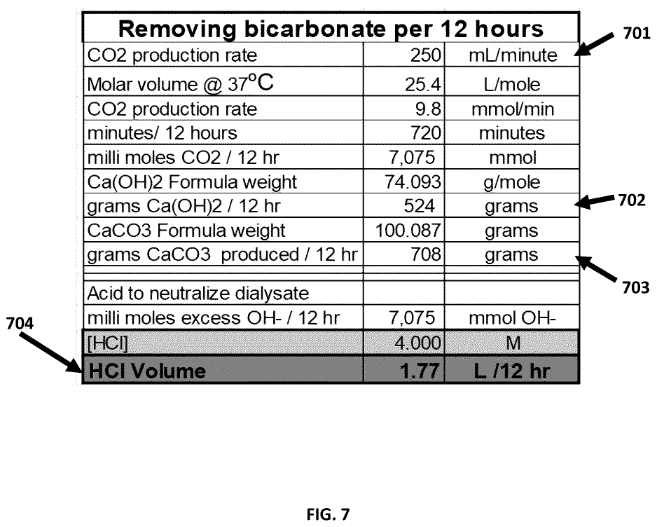

FIG. 7 shows the stoichiometry of Ca(OH)₂ for HCO₃⁻ removal from dialysate at 100% of basal CO₂ generation rate, for 12 hours. A body CO₂ production rate at rest is 250 ml/min (9.8 mmol/min). At a serum bicarbonate of 40 mEq/L this rate passes from blood to dialysate at flowrates of 250 mL/min each (See 701). Removing all of the HCO₃⁻ would require 524 grams of Ca(OH)₂ every 12 hours (See 702), which creates 708 grams of CaCO₃ (See 703). An HCl infusion of 1.77 L of 4 M HCl solution would be required to balance pH changes (See 704).

The system and method of the present disclosure is a practical, reliable and safe in dialysate regeneration use. The system 1 can provide HCO₃⁻ removal at rate that matches total body CO₂ generation at rest. Provides Cl⁻ and OH⁻ to replace removed HCO₃⁻ without adding cations to dialysate such as Na⁺ or Ca⁺⁺. Must be designed to not infuse more Ca(OH)₂ than needed to remove all HCO₃⁻, or ionized Ca⁺⁺ will increase in dialysate. The system can use standard components of a CVVHD system. Use of an Outside-In dialyzer can help avoid need for anticoagulants and provide dialysis for several days without clotting. The system may be an effective ECCO₂R system and avoid use of positive-pressure ventilation in many patients with respiratory failure, including those with pneumonia, ARDS, and Covid.

The system and each of the subsystems may interact with one or more devices and networks to communicate with the client(s) and each other. The various systems of the apparatus 1 can include transceivers for communication. One or more processors can be utilized to control the system 1. The various systems disclosed can be executed via one ore more programs, including a first measurement program, a calcium hydroxide treatment program, a mixing program, a volume control program, a safety monitor program, and pH program to execute one or more of the system routines. The various systems can similarly be communicatively coupled to a remote controller apparatus such as a computer or server. Similarly, the system may be communicatively coupled to a display for monitoring during the process. Hardware may include video cameras, microphones, video players, cameras, and accessing other apps or software for communicating and transferring data to update the display output. The output may then be modified with hardware data capture information. Such as using the camera to capture video and displaying the video in real-time on the output display, including audio played over speakers.

Other networks may be in communication with the system, such as a network of service providers and information related to providing services. Other networks and other applications, such as accessing a calendar to determine availability and scheduling the service appointment. Navigation may be accessed to help with directions to where the service is to be provided.

The client may provide an interface to other devices including, without limitation, servers, other clients, printers, database servers, print servers, file servers, communication servers, distributed servers, and the like. Additionally, this coupling and/or connection may facilitate remote execution of program across the network. The networking of some or all of these devices may facilitate parallel processing of a program or method at one or more location. In addition, any of the devices attached to the client through an interface may include at least one storage medium capable of storing methods, programs, applications, code, and/or instructions. A central repository may provide program instructions to be executed on different devices. In this implementation, the remote repository may act as a storage medium for program code, instructions, and programs.

The methods and systems described herein may be deployed in part or in whole through network infrastructures. The network infrastructure may include elements such as computing devices, servers, routers, hubs, firewalls, clients, personal computers, communication devices, routing devices and other active and passive devices, modules and/or components as known in the art. The computing and/or non-computing device(s) associated with the network infrastructure may include, apart from other components, a storage medium such as flash memory, buffer, stack, RAM, ROM, and the like. The processes, methods, program codes, instructions described herein and elsewhere may be executed by one or more of the network infrastructural elements.

The methods, program codes, and instructions described herein and elsewhere may be implemented on a cellular network having multiple cells. The cellular network may either be frequency division multiple access (FDMA) network or code division multiple access (CDMA) network. The cellular network may include mobile devices, cell sites, base stations, repeaters, antennas, towers, and the like. The cell network may be a GSM, GPRS, 3G, EVDO, mesh, or other networks types.

The methods, programs codes, and instructions described herein and elsewhere may be implemented on or through mobile devices. The mobile devices may include navigation devices, cell phones, mobile phones, mobile personal digital assistants, laptops, palmtops, netbooks, pagers, electronic books readers, music players, and the like. These devices may include, apart from other components, a storage medium such as a flash memory, buffer, RAM, ROM and one or more computing devices. The computing devices associated with mobile devices may be enabled to execute program codes, methods, and instructions stored thereon. Alternatively, the mobile devices may be configured to execute instructions in collaboration with other devices. The mobile devices may communicate with base stations interfaced with servers and configured to execute program codes. The mobile devices may communicate on a peer-to-peer network, mesh network, or other communications network. The program code may be stored on the storage medium associated with the server and executed by a computing device embedded within the server. The base station may include a computing device and a storage medium. The storage device may store program codes and instructions executed by the computing devices associated with the base station.

The computer software, program codes, and/or instructions may be stored and/or accessed on machine readable media that may include: computer components, devices, and recording media that retain digital data used for computing for some interval of time; semiconductor storage known as random access memory (RAM); mass storage typically for more permanent storage, such as optical discs, forms of magnetic storage like hard disks, tapes, drums, cards and other types; processor registers, cache memory, volatile memory, non-volatile memory; optical storage such as CD, DVD; removable media such as flash memory (e.g. USB sticks or keys), floppy disks, magnetic tape, paper tape, punch cards, standalone RAM disks, Zip drives, removable mass storage, off-line, and the like; other computer memory such as dynamic memory, static memory, read/write storage, mutable storage, read only, random access, sequential access, location addressable, file addressable, content addressable, network attached storage, storage area network, bar codes, magnetic ink, and the like.

While the invention has been described above in terms of specific embodiments, it is to be understood that the invention is not limited to these disclosed embodiments. Upon reading the teachings of this disclosure many modifications and other embodiments of the invention will come to mind of those skilled in the art to which this invention pertains, and which are intended to be and are covered by both this disclosure and the appended claims. It is indeed intended that the scope of the invention should be determined by proper interpretation and construction of the appended claims and their legal equivalents, as understood by those of skill in the art relying upon the disclosure in this specification and the attached drawings. Various related embodiments of the invention are also described in Provisional patent Appendix A, which is incorporated herein by reference in its entirety.

While the invention has been described above in terms of specific embodiments, it is to be understood that the invention is not limited to these disclosed embodiments. Upon reading the teachings of this disclosure many modifications and other embodiments of the invention will come to mind of those skilled in the art to which this invention pertains, and which are intended to be and are covered by both this disclosure and the appended claims. It is indeed intended that the scope of the invention should be determined by proper interpretation and construction of the appended claims and their legal equivalents, as understood by those of skill in the art relying upon the disclosure in this specification and the attached drawings.

What is claimed is:

1. A method of removing bicarbonate from a patient's blood, comprising the steps of:
providing a bicarbonate removal system comprising:
a dialyzer, a CO2 measurement system, a calcium hydroxide system, a mixer, a reactor, a volume control pump, acid pump system, and a pH detector;
pumping a dialysate through the bicarbonate removal system using a first pump, wherein a second pump provides a first specimen of the dialysate to the CO2 measurement system, wherein the CO2 measurement system provides a CO2 measurement of the dialysate to the calcium hydroxide system, wherein the remaining dialysate is pumped into a mixer;
providing a prescribed amount of calcium hydroxide from the calcium hydroxide system into the mixer based upon the CO2 measurement;
providing a reactor to continuously mix the dialysate and the calcium hydroxide until a calcium carbonate precipitate forms;
pumping the dialysate through a filter of the reactor to remove any unreacted calcium hydroxide and calcium carbonate precipitate;
measuring the pH of the dialysate to determine a pH reading with the pH detector;
providing an acid to reduce pH of the dialysate if the pH reading is 12.5 or higher;
pumping the dialysate back into the dialyzer and out to the patient.

2. The method of claim 1, wherein the mixer is a static mixer.

3. The method of claim 1, wherein reactor provides a solids separator after being mixed within the static mixer.

4. The method of claim 3, wherein the volume control pump and a waste reservoir are part of the reactor.

5. The method of claim 1, wherein a HCl solution is pumped into the dialysate after the influx dialysate, and the Ca(OH)₂ suspension are mixed and before the dialysate is returned to the dialyzer.

6. The method of claim 1, wherein the bicarbonate removal system further includes a safety sample pump that pumps a second specimen of the dialysate to a calcium sensor and a carbon dioxide sensor configured to monitor the calcium content and carbon dioxide content after the dialysate after it passes through the reactor and filter.

7. The method of claim 6, wherein the CO₂ measurement system further comprises a CO₂ electrode and pH electrode.

8. The method of claim 7, wherein the CO₂ measurement system further comprises an acid reservoir and an acid pump, wherein the acid pump can provide an acid from the acid reservoir to the sample provided to the CO₂ measurement system.

9. The method of claim 9, wherein the acid is HCL.

10. A system for removing bicarbonate from dialysate, comprising:
a dialyzer having an inlet and an outlet, further comprising one or more conduits providing a loop between the inlet and outlet for the dialysate to flow;
a dialysate pump positioned after the dialyzer configured to pump the dialysate through the loop;
a sample pump configured to remove a pre-determined amount of dialysate from the dialyzer loop, wherein the sample pump is communicatively coupled to the CO₂ measurement system, wherein the CO2 measurement system can be used to determine the total CO2 in the dialysate;
a calcium hydroxide dispensing system, wherein the calcium hydroxide dispensing system includes a calcium hydroxide pump and a hopper portion, wherein the hopper portion can store a calcium hydroxide powder or suspension at a pre-determined concentration, wherein the calcium hydroxide dispensing system is communicatively coupled to the CO2 measurement system;
a mixer fluidly coupled to the calcium hydroxide dispensing system, wherein the dialysate is pumped to a reactor with a mixer to vigorously mix the dialysate and the calcium hydroxide;
a volume control system comprising a volume control pump and a volume waste reservoir;
a safety monitor system comprising one or more sensors;
a pH sensor to obtain an outlet pH of the dialysate; and
an acid pump system communicatively coupled to the pH sensor to provide an acid to the dialysate when the dialysate is at or above a prescribed pH.

11. The system of claim 10, wherein the acid provided by the acid pump is HCL.

12. The system of claim 11, wherein the CO₂ measurement system further comprises a first CO₂ electrode, first pH electrode, a first acid reservoir, a first acid pump, and a waste reservoir.

13. The system of claim 12, wherein calcium hydroxide dispensing system is communicative and fluidly coupled to the calcium hydroxide pump.

14. The system of claim 13, wherein the prescribed pH of dialysate returning to the dialyzer is 12.5.

15. The system of claim 14, wherein the safety monitor system comprises a CO₂ electrode and a calcium electrode.

16. The system of claim 15, further comprising a leak detector configured to monitor for blood of the dialysate travels through the loop positioned between the dialysate pump and calcium hydroxide dispensing system.

17. The system of claim 16, wherein the calcium hydroxide is comprised of a calcium hydroxide solution.

18. The system of claim 17, wherein the dialyzer is an Outside-in dialyzer.

19. The system of claim 18, wherein the safety monitor system includes an acid reservoir and an acid pump.

20. The system of claim 19, wherein the acid pump system provides acid to the dialysate until it is at or below pH10.
